(19) United States
(12) Patent Application Publication
(10) Pub. No.: US 2023/0137017 A1
(43) Pub. Date: May 4, 2023

(54) OPTICAL ACTIVITY DETECTING DEVICE

(71) Applicant: Lextar Electronics Corporation, Hsinchu (TW)

(72) Inventor: Fu-Han HO, Hsinchu (TW)

(21) Appl. No.: 17/868,689

(22) Filed: Jul. 19, 2022

(30) Foreign Application Priority Data
Nov. 2, 2021 (TW) 110140706

(51) Int. Cl. G01S 7/499; G02B 27/28; G01S 17/04; G01S 7/481

(52) U.S. Cl. CPC G01S 7/499; G02B 27/28; G01S 17/04; G01S 7/4811

(57) ABSTRACT

An optical activity detecting device is provided. The optical activity detecting device is adapted to detect an object. The optical activity detecting device includes a light source, a filter, a first polarizer, a second polarizer, a first compensation film and a first detector. The light source provides a light beam. The light beam travels from the light source, passes through the filter and the first polarizer, and enters the object. At least a portion of the light beam travels from the object, passes through the second polarizer and the first compensation film and is received by the first detector.

### CROSS REFERENCE TO RELATED APPLICATIONS

This application claims priority of Taiwan Patent Application No. 110140706, filed on Nov. 2, 2021, the entirety of which is incorporated by reference herein.

### BACKGROUND OF THE INVENTION

### Field of the Invention

The present invention relates to a detecting device, and in particular to an optical activity detecting device.

### Description of the Related Art

Optical activity detecting technology can be utilized to blood glucose test.

Conventional optical activity detecting device has a light source, and an optical analyzer, which detects the eye of the testee. The conventional light source and the optical analyzer have complex structure and increased cost, and the conventional optical activity detecting device is thus huge and not easy to move, which cannot realize continuous non-invasive detection.

### BRIEF SUMMARY OF THE INVENTION

Embodiments of the invention are provided to address the aforementioned difficulty.

In one embodiment, an optical activity detecting device is provided. The optical activity detecting device is adapted to detect an object. The optical activity detecting device includes a light source, a filter, a first polarizer, a second polarizer, a first compensation film and a first detector. The light source provides a light beam. The light beam travels from the light source, passes through the filter and the first polarizer, and enters the object. At least a portion of the light beam travels from the object, passes through the second polarizer and the first compensation film and is received by the first detector.

In one embodiment, the phase difference between the first polarizer and the second polarizer is 90 degrees or 0 degrees.

In one embodiment, the optical activity detecting device further comprises a substrate and a first spacer, the light source and the first detector are disposed on the substrate, and the first spacer is disposed between the light source and the first detector.

In one embodiment, the optical activity detecting device further comprises a third polarizer, a second compensation film and a second detector. At least a portion of the light beam travels from the object, passes through the third polarizer and the second compensation film, and is received by the second detector.

In one embodiment, the phase difference between the first polarizer and the second polarizer is 90 degrees, and the phase difference between the first polarizer and the third polarizer is 0 degrees.

In one embodiment, the optical activity detecting device further comprises a substrate and a first spacer, wherein the light source, the first detector and the second detector are disposed on the substrate, and the first spacer is disposed between the light source and the first detector.

In one embodiment, the optical activity detecting device further comprises a second spacer, wherein the second spacer is disposed between the first detector and the second detector.

In one embodiment, the optical activity detecting device is a wearable device, and the light source, the filter, the first polarizer, the second polarizer, the first compensation film and the first detector are integrated into on single module package.

In one embodiment, the light source comprises an edge-emitting semiconductor laser source, a vertical cavity surface emitting laser source, light-emitting diodes of organic or inorganic materials without specific polarization characteristics, a near infrared light source or an infrared light source.

In one embodiment, the first detector comprises Si, GaAs or InGaAs.

In one embodiment, the first polarizer and the second polarizer comprise organic polymer materials or inorganic crystal materials.

In one embodiment, the first compensation film comprises liquid-crystal polymer (LCP).

In one embodiment, the filter corresponds to a particular wavelength half-width.

In one embodiment, the optical activity detecting device further comprises a first lens unit and a second lens unit, wherein the light beam travels from the light source, passes through the filter, the first polarizer and the first lens, and enters the object, and at least a portion of the light beam travels from the object, passes through the second lens unit, the second polarizer and the first compensation film, and is received by the first detector.

In one embodiment, the first lens comprises a concave lens, and the second lens comprises a convex lens.

In one embodiment, an optical activity detecting device is provided. The optical activity detecting device includes a light source, a first polarizer, a second polarizer and a first detector. The light source provides a light beam. The light beam travels from the light source, passes through the first polarizer, and enters the object. The phase difference between the first polarizer and the second polarizer is 90 degrees or 0 degrees. At least a portion of the light beam travels from the object, passes through the second polarizer and is received by the first detector.

In the embodiment of the invention, the optical activity detecting device can be a wearable device, and is wear on the head of the testee. The light source, the filter, the first polarizer, the second polarizer, the first compensation film and the first detector are integrated into on single module package. The optical activity detecting device of the embodiment of the invention has simpler structure, small size and decreased cost, which can be carried easily, and can realize continuous non-invasive detection.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1 shows an optical activity detecting device of a first embodiment of the invention;

FIG. 2 shows an optical activity detecting device of a second embodiment of the invention;

FIG. 3 shows an optical activity detecting device of a third embodiment of the invention; and

FIG. 4 shows an optical activity detecting device of a fourth embodiment of the invention.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

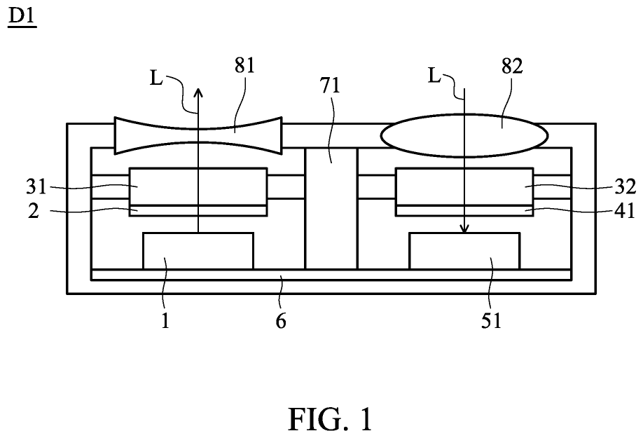

FIG. 1 shows an optical activity detecting device of a first embodiment of the invention. With reference to FIG. 1, the optical activity detecting device D1 of the first embodiment of the invention is adapted to detect an object (not shown). The optical activity detecting device D1 includes a light source 1, a filter 2, a first polarizer 31, a second polarizer 32, a first compensation film 41 and a first detector 51. The light source 1 provides a light beam L. The light beam L travels from the light source 1, passes through the filter 2 and the first polarizer 31, and enters the object (not shown). At least a portion of the light beam L travels from the object (not shown), passes through the second polarizer 32 and the first compensation film 41 and is received by the first detector 51.

With reference to FIG. 1, in one embodiment, the phase difference between the first polarizer 31 and the second polarizer 32 is 90 degrees or 0 degrees. However, the disclosure is not meant to restrict the invention. In other embodiment, the phase difference between the first polarizer 31 and the second polarizer 32 can be modified. For example, the phase difference between the first polarizer 31 and the second polarizer 32 can be 45 degrees.

In one embodiment, the phase difference between the first polarizer 31 and the second polarizer 32 is 90 degrees. When there is specific rotation, the light intensity is increased with the specific rotation.

In other embodiment, the phase difference between the first polarizer 31 and the second polarizer 32 is 0 degrees. When there is specific rotation, the light intensity is decreased with the specific rotation.

With reference to FIG. 1, in one embodiment, the optical activity detecting device further comprises a substrate 6 and a first spacer 71. The light source 1 and the first detector 51 are disposed on the substrate 6. The first spacer 71 is disposed between the light source 1 and the first detector 51.

In one embodiment, the light source 1 comprises an edge-emitting semiconductor laser source, a vertical cavity surface emitting laser source, light-emitting diodes of organic or inorganic materials without specific polarization characteristics, a near infrared light source or an infrared light source.

In one embodiment, the first detector 51 includes Si, GaAs or InGaAs.

In one embodiment, the first polarizer 31 and the second polarizer 32 include organic polymer materials or inorganic crystal materials.

In one embodiment, the first compensation film 41 includes liquid-crystal polymer (LCP). The compensation film compensates the phase deflection or the optical activity of the optical system.

In one embodiment, the filter is utilized to purify wavelength (reducing half-width).

With reference to FIG. 1, in one embodiment, the optical activity detecting device D1 further comprises a first lens unit 81 and a second lens unit 82. The light beam travels L from the light source 1, passes through the filter 2, the first polarizer 31 and the first lens 81, and enters the object (not shown), and at least a portion of the light beam L travels from the object (not shown), passes through the second lens unit 82, the second polarizer 32 and the first compensation film 41, and is received by the first detector 51.

With reference to FIG. 1, in one embodiment, the first lens 81 includes a concave lens to homogenize light distribution. The second lens 82 includes a convex lens to collect light. The disclosure is not meant to restrict the invention.

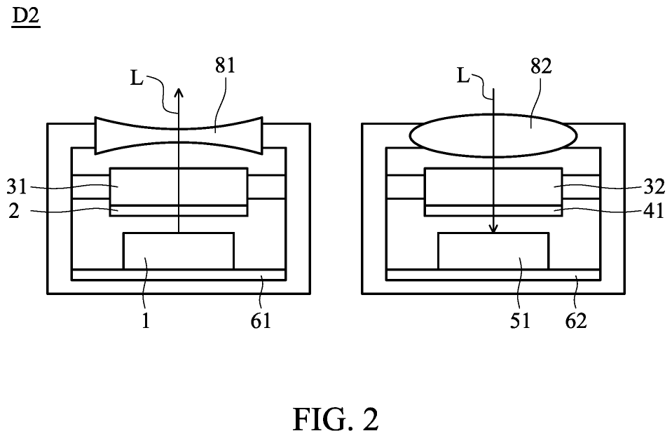

FIG. 2 shows an optical activity detecting device of a second embodiment of the invention. With reference to FIG. 2, in this embodiment, the light source 1 is disposed on a first substrate 61, and the first detector 51 is disposed on a second substrate 62. In the embodiment of the invention, the light source unit (including the light source 1, the filter 2, the first polarizer 31 and the first lens 81) and the detection unit (including the second lens 82, the second polarizer 32, the first compensation film 41 and the detector 51) can be integrated in one single device housing (the first embodiment) or disposed in different device housings (the second embodiment). The disclosure is not meant to restrict the invention.

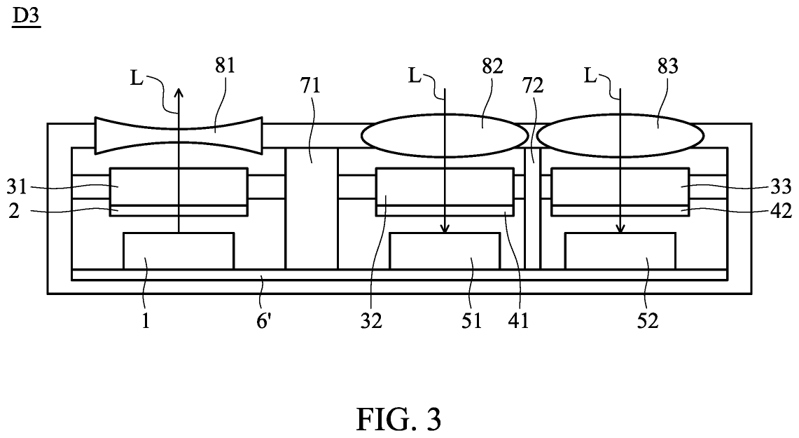

FIG. 3 shows an optical activity detecting device of a third embodiment of the invention. With reference to FIG. 3, in this embodiment, the optical activity detecting device D3 further comprises a third lens 83, a third polarizer 33, a second compensation film 42 and a second detector 52. At least a portion of the light beam L travels from the object (not shown), passes through the third lens 83, the third polarizer 33 and the second compensation film 42, and is received by the second detector 52.

With reference to FIG. 3, in one embodiment, the phase difference between the first polarizer 31 and the second polarizer 32 is 90 degrees, and the phase difference between the first polarizer 31 and the third polarizer 33 is 0 degrees. Therefore, the detected results of the first detector 51 and the second detector 52 can be cross reference.

With reference to FIG. 3, in one embodiment, the optical activity detecting device further comprises a substrate 6′ and a first spacer 71. The light source 1, the first detector 51 and the second detector 52 are disposed on the substrate 6′. The first spacer 71 is disposed between the light source 1 and the first detector 51.

With reference to FIG. 3, in one embodiment, the optical activity detecting device D3 further comprises a second spacer 72. The second spacer 72 is disposed between the first detector 51 and the second detector 52.

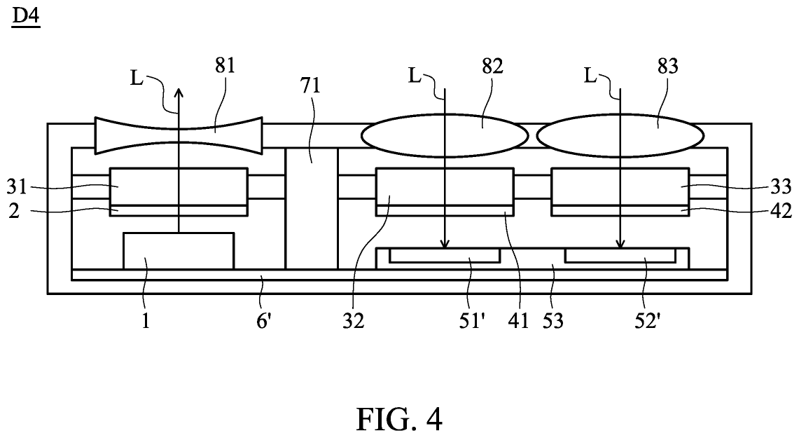

FIG. 4 shows an optical activity detecting device of a fourth embodiment of the invention. With reference to FIG. 4, in this embodiment, the first detector 51′ and the second detector 52′ of the optical activity detecting device D4 are integrated into a detector array module 53.

In one embodiment, the object can be an eyeball. The sizes of the light source, the filter, the first polarizer, the second polarizer, the first compensation film and the first detector are much smaller than the size of the object.

In the embodiment of the invention, the optical activity detecting device can be a wearable device, and is wear on the head of the testee. The light source, the filter, the first polarizer, the second polarizer, the first compensation film and the first detector are integrated into on single module package. The optical activity detecting device of the embodiment of the invention has simpler structure, small size and decreased cost, which can be carried easily, and can realize continuous non-invasive detection.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having the same name (but for use of the ordinal term).

While the invention has been described by way of example and in terms of the preferred embodiments, it should be understood that the invention is not limited to the disclosed embodiments. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. An optical activity detecting device, adapted to detect an object, comprising:
a light source, providing a light beam;
a filter;
a first polarizer, wherein the light beam travels from the light source, passes through the filter and the first polarizer, and enters the object;
a second polarizer;
a first compensation film; and
a first detector, wherein at least a portion of the light beam travels from the object, passes through the second polarizer and the first compensation film and is received by the first detector.

2. The optical activity detecting device of claim 1, wherein a phase difference between the first polarizer and the second polarizer is 90 degrees or 0 degrees.

3. The optical activity detecting device of claim 1, further comprising a substrate and a first spacer, the light source and the first detector are disposed on the substrate, and the first spacer is disposed between the light source and the first detector.

4. The optical activity detecting device of claim 1, further comprising:
a third polarizer;
a second compensation film; and
a second detector, wherein at least a portion of the light beam travels from the object, passes through the third polarizer and the second compensation film, and is received by the second detector.

5. The optical activity detecting device of claim 4, wherein the phase difference between the first polarizer and the second polarizer is 90 degrees, and a phase difference between the first polarizer and the third polarizer is 0 degrees.

6. The optical activity detecting device of claim 4, further comprising a substrate and a first spacer, wherein the light source, the first detector and the second detector are disposed on the substrate, and the first spacer is disposed between the light source and the first detector.

7. The optical activity detecting device of claim 6, further comprising a second spacer, wherein the second spacer is disposed between the first detector and the second detector.

8. The optical activity detecting device of claim 1, wherein the optical activity detecting device is a wearable device, and the light source, the filter, the first polarizer, the second polarizer, the first compensation film and the first detector are integrated into on single module package.

9. The optical activity detecting device of claim 1, wherein the light source comprises an edge-emitting semiconductor laser source, a vertical cavity surface emitting laser source, light-emitting diodes of organic or inorganic materials without specific polarization characteristics, a near infrared light source or an infrared light source.

10. The optical activity detecting device of claim 1, wherein the first detector comprises Si, GaAs or InGaAs.

11. The optical activity detecting device of claim 1, wherein the first polarizer and the second polarizer comprise organic polymer materials or inorganic crystal materials.

12. The optical activity detecting device of claim 1, wherein the first compensation film comprises liquid-crystal polymer (LCP).

13. The optical activity detecting device of claim 1, wherein the filter corresponds to a particular wavelength half-width.

14. The optical activity detecting device of claim 1, further comprising a first lens unit and a second lens unit, wherein the light beam travels from the light source, passes through the filter, the first polarizer and the first lens, and enters the object, and at least a portion of the light beam travels from the object, passes through the second lens unit, the second polarizer and the first compensation film, and is received by the first detector.

15. The optical activity detecting device of claim 14, wherein the first lens comprises a concave lens, and the second lens comprises a convex lens.

16. An optical activity detecting device, adapted to detect an object, comprising:
a light source, providing a light beam;
a first polarizer, wherein the light beam travels from the light source, passes through the first polarizer, and enters the object;
a second polarizer, wherein the phase difference between the first polarizer and the second polarizer is 90 degrees or 0 degrees; and
a first detector, wherein at least a portion of the light beam travels from the object, passes through the second polarizer and is received by the first detector.

17. The optical activity detecting device of claim 16, further comprising:
a third polarizer;
a second detector, wherein at least a portion of the light beam travels from the object, passes through the third polarizer, and is received by the second detector.

18. The optical activity detecting device of claim 17, wherein the phase difference between the first polarizer and the second polarizer is 90 degrees, and the phase difference between the first polarizer and the third polarizer is 0 degrees.
